(19) United States
(12) Patent Application Publication
(10) Pub. No.: US 2026/0083490 A1
(43) Pub. Date: Mar. 26, 2026

(54) METHOD AND DEVICE FOR ABLATING ANATOMICAL TARGETS FROM WITHIN A BLOOD VESSEL WITH IN-PROCEDURE FEEDBACK

(71) Applicant: Steer Medical Inc., Quebec (CA)

(72) Inventors: Dan Wittenberger, Quebec (CA); Domenic Santoianni, Quebec (CA); Darrin J. Kent, Murrieta, CA (US); Michael J. Froncioni, Quebec (CA); Alan Clack, Quebec (CA)

(21) Appl. No.: 19/337,355

(22) Filed: Sep. 23, 2025

Related U.S. Application Data
(60) Provisional application No. 63/698,356, filed on Sep. 24, 2024.

(51) Int. Cl. A61B 18/02; A61B 18/00; A61B 90/00; A61M 25/10

(52) U.S. Cl. CPC A61B 18/02; A61B 90/37; A61M 25/1002; A61M 25/1011; A61B 2018/00404; A61B 2018/00434; A61B 2018/00577; A61B 2018/00797; A61B 2018/0212; A61B 2018/0262; A61B 2090/378; A61M 2025/1052; A61M 2025/1068; A61M 2205/3327; A61M 2205/3331; A61M 2205/3368; A61M 2205/3375; A61M 2205/3606; A61M 2205/502; A61M 2205/583; A61M 2210/12

(57) ABSTRACT

A cryo-ablation system treats conditions associated with sympathetic nervous system hyperactivity using a catheter with a configurable heat transfer element that transitions from a flat delivery profile to deployed configurations including helical shapes. The system includes deployable temperature probes that directly measure tissue temperature at target locations inside or outside blood vessels. A computational control system processes temperature data from multiple sensors and deployable probes to calculate real-time lethal isotherm progression using heat transfer modeling algorithms. The system provides vessel occlusion during treatment and real-time feedback for treatment optimization. Methods include introducing the catheter into a renal artery, deploying the heat transfer element, occluding the vessel, deploying temperature probes to target locations within or outside the vessel, delivering cryogenic temperatures while monitoring probe temperatures, and terminating treatment based on computational analysis of temperature data to achieve target ablation depths.

### CROSS-REFERENCE TO RELATED APPLICATION

This application claims the benefit of U.S. Provisional Application No. 63/698,356 filed Sep. 24, 2024.

### FIELD OF THE INVENTION

The present invention relates to medical devices and methods for treating hypertension and other conditions associated with sympathetic nervous system hyperactivity, specifically through cryo-ablation of anatomical targets adjacent to blood vessels using occlusive catheter-based systems with real-time monitoring capabilities and deployable temperature sensing probes.

### BACKGROUND OF THE INVENTION

Resistant hypertension is a medical condition where blood pressure remains uncontrolled despite medical therapy with multiple antihypertensive medications. The condition has been linked to hyperactivity of the sympathetic nervous system, particularly the renal sympathetic nerves that innervate the kidneys. These nerves regulate blood pressure through multiple mechanisms including renin release, sodium reabsorption, and renal blood flow.

Renal denervation has been developed as an interventional treatment for resistant hypertension, targeting the sympathetic nerve fibers that surround the renal arteries. Current renal denervation techniques include radiofrequency ablation, focused ultrasound, neurolytics, and cryoablation methods.

Current radiofrequency ablation operates by delivering thermal energy to denature proteins and destroy nerve tissue. These heat-based methods can cause thermal damage to the arterial wall, potentially leading to scarring and vessel narrowing. To reduce stenosis risk, energy delivery is controlled and spatially distributed, often in discrete patterns spaced around the vessel circumference. The discrete treatment pattern may leave nerve fibers intact between ablation points. Continuous blood flow through the artery during treatment acts as a heat sink, dissipating thermal energy and affecting the depth and consistency of ablation.

Focused ultrasound approaches have been developed to deliver energy circumferentially and penetrate deeper into the tissue. Ultrasound methods face challenges with energy delivery control and may cause heating of non-target tissues.

Cryoablation approaches for renal denervation have been described in the prior art and demonstrated clinically. Simple cryoablation catheters have been used to create discrete ablation lesions in renal arteries. Current cryoablation systems lack comprehensive real-time monitoring capabilities to assess treatment depth and progression. Existing systems do not provide direct tissue temperature measurement at the target ablation sites outside the vessel wall. Prior cryoablation approaches do not incorporate comprehensive vessel occlusion strategies to eliminate heat sink effects. Current systems lack integrated computational algorithms for real-time calculation of lethal isotherm progression.

Current methods, including basic cryoablation approaches, lack comprehensive real-time feedback mechanisms to guide treatment and confirm efficacy. Operators rely on indirect measures and post-procedural assessments, making it difficult to optimize treatment parameters during the procedure or ensure adequate nerve destruction at the target locations outside the vessel wall.

### BRIEF SUMMARY OF THE INVENTION

The present invention provides a cryo-ablation system and method for the treatment of resistant hypertension through the ablation of renal sympathetic nerves. The system includes a cryo-ablation treatment element configured to be placed within a blood vessel and a temperature probe outside the cryo-ablation catheter, to perform direct temperature measurement of the tissue, at a known distance from the balloon.

The system can include an occlusive cryo-ablation balloon for occluding a blood vessel to reduce heat load and increase efficiency. In one configuration, the occlusive balloon can be selectively transitionable from a substantially flat configuration to a cylindrical, conical, or helical shape. Lesions can be created that are continuous, circumferential, or helical elongated, and the cryogenic treatment region can be at least 5 mm in length. Radiopaque markers can be integrated into the catheter to indicate the position of the balloon, temperature probe, and catheter tip.

A temperature monitoring system is provided to measure temperatures at multiple locations, including at least one location outside the balloon; and including at least one location that can be positioned within or outside of the vasculature.

A pressure sensing system is provided to detect arterial occlusion. An ultrasound imaging system can be used to visualize progression of tissue freezing, measure a diameter of the frozen volume, and calculate a position of a lethal isotherm to provide real-time feedback during a cryo-ablation procedure.

A method for treating resistant hypertension is provided that includes introducing a cryo-ablation catheter of the invention into a renal artery; transitioning the cryo-ablation balloon from a flat configuration to a cylindrical, conical, or helical configuration within the renal artery to create a continuous cryogenic treatment region; occluding the renal artery using the cryo-ablation balloon to reduce blood flow and increase the efficacy of the cryo-ablation; and applying cryogenic temperatures to ablate the renal sympathetic nerves adjacent to the renal artery.

Tissue temperature is measured at a known distance, outside the cryo-ablation balloon; the progression of tissue freezing is monitored and the cryo-ablation parameters are adjusted based on real-time feedback from temperature measurements and/or ultrasound imaging. A pressure sensing system confirms arterial occlusion distal to the cryo-ablation balloon.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 illustrates a cryo-ablation catheter in a delivery configuration with the treatment element in a substantially flat profile for navigation through the vasculature;

FIG. 2 depicts the catheter of FIG. 1 with the treatment element deployed in a helical configuration creating an elongated treatment zone;

FIG. 3 shows the treatment element in an alternative helical configuration with adjustable pitch and diameter, demonstrating the device's adaptability to different vessel sizes;

FIG. 4 illustrates an embodiment including a compliant occlusion balloon positioned proximal to the treatment element for enhanced vessel occlusion;

FIG. 5 provides a cross-sectional view showing temperature probe placement and the spatial relationship between measured temperatures (T0, T1) and the calculated lethal isotherm distance;

FIG. 6 presents a flowchart of the temperature monitoring and control method, showing the relationship between probe temperatures, heat transfer calculations, and treatment endpoint determination;

FIG. 7 depicts the deployable temperature probe system for direct tissue temperature measurement, showing the probe extending through the vessel wall into the perivascular space;

FIG. 8 shows a catheter handle assembly including deflection mechanism, connectors, and treatment element controls;

FIG. 9 is a detailed view of the treatment element with integrated temperature probe in the deployed position;

FIG. 10 depicts a non-deflectable catheter embodiment with a simplified manifold handle design;

FIG. 11 shows a high-pressure catheter embodiment with rigid tip construction capable of utilizing high-pressure refrigerant systems;

FIG. 12 illustrates the high-pressure catheter with an optional inflatable balloon covering the treatment element;

FIG. 13 depicts a high-pressure catheter system with proximal occlusion balloon for enhanced blood flow control; and

FIG. 14 shows an embodiment where refrigerant circulates through a continuous helical tube configuration for distributed cooling.

### DETAILED DESCRIPTION

The present invention provides a cryo-ablation system for percutaneous treatment of anatomical targets adjacent to blood vessels. The system employs temperature sensing, computational algorithms relating to the sensed temperatures, and catheter features that enable control of treatment delivery and real-time assessment of therapeutic endpoints.

As discussed in more detail below, the system includes a cryoablation catheter with a heat transfer element mounted proximal to the distal end of the catheter. The element is configured to occlude a blood vessel and deliver cryo-energy to an anatomical target outside the blood vessel. The element may also be non-occlusive, but may be used in conjunction with a proximal occlusive element, such as a balloon. A refrigerant delivery and control unit (cryo-console) supplies refrigerant to the ablation device and controls the safe flow and recovery thereof. The catheter is an elongated polymer and optionally metal reinforced structure with a proximal connection (hub) allowing refrigerant supply and removal. The hub can provide guidewire passage through a dedicated lumen. The guidewire lumen can, in certain embodiments, be actuated by the user causing an axial and/or a rotational movement.

The heat transfer element is a tubular structure connected to the distal end of the shaft and wrapped around the guidewire lumen. In another embodiment the heat transfer element is a compliant or semi-compliant balloon with a substantially cylindrical or conical profile. In yet another embodiment, the heat transfer element is a metallic structure and may be expandable by the user, or self-expanding. In yet another embodiment, the heat transfer element is a composite structure, comprised of a metallic frame and polymeric coating. A refrigerant supply tube runs through the length of the catheter shaft and ends, along with the tubular structure or the balloon in the atraumatic tip at the distal end of the guidewire lumen. The refrigerant supply tube has one or a multitude of ports along the length of the heat transfer element. When refrigerant is delivered, the heat transfer element serves as an expansion chamber for the refrigerant undergoing a liquid to gas phase change and/or Joule-Thomson effect and removes heat from the contacting tissue (delivers cryo-energy). The pressure created by the refrigerant causes the tubular structure to inflate. It is configured to create a fully circular, elongated ablation zone, or, by moving the guidewire lumen and varying the pitch of the coil, a helical ablation zone. Typical ablation length is in the range of 5-40 mm, while the diameter of the tubular structure is about 2 mm. By rotating the guidewire lumen, the outer diameter of the coil can be reduced or increases to fit the vessel diameter, in the range of 3-8 mm. Alternatively, a liquid refrigerant is circulated to the cooling element and returned as a liquid or as a gas/liquid mix. The system includes means to assess vessel occlusion, means to limit mechanical stress to the vessel walls, and means to intraoperatively assess progression of the cryo-treatment by direct temperature monitoring in the tissue.

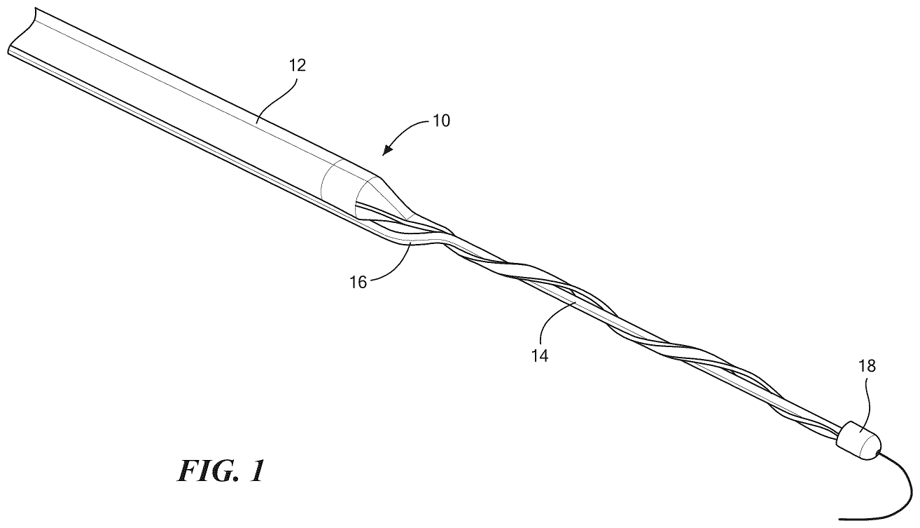

Referring now to FIG. 1, an exemplary cryo-ablation catheter 10 features a multi-lumen construction for refrigerant delivery, temperature monitoring including deployable probe systems, pressure monitoring, and device navigation capabilities as is known in the art. The catheter 10 comprises a proximal shaft portion 12, an elongated distal shaft 14 with a flexible polymer body and optional metallic reinforcement, providing pushability, torqueability, and flexibility for vascular navigation while accommodating the internal lumen arrangement required for monitoring systems. A heat transfer (treatment) element 16 is shown in a delivery configuration having a substantially flat profile suitable for navigation through the vasculature. The catheter 10 incorporates an atraumatic tip 18 at the distal end of the shaft 14 to facilitate vascular navigation. The catheter 10 incorporates thermal insulation along its length to prevent heat gain during refrigerant transit and maintain cryogenic energy delivery to the treatment site. The lumen configuration discussed in more detail below minimizes cross-sectional area while maintaining flow capacity and accommodating the deployable probe mechanisms. A proximal hub discussed below provides connections for refrigerant supply and return lines, electrical connections for temperature or other sensors, deployable probe controls, and optional guidewire access as is known in the art.

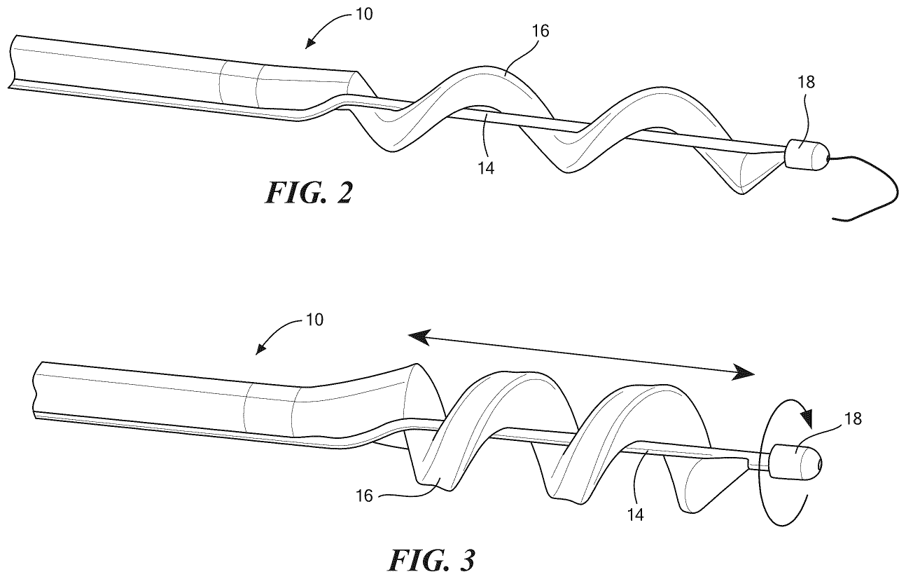

FIG. 2 demonstrates the transformation capability of the heat transfer element 16 of FIG. 1 from its relatively flat, minimal diameter delivery configuration shown to an expanded, greater diameter, active treatment configuration. The heat transfer element 16 is shown deployed in a helical configuration that creates an elongated treatment zone for placement along the length of a renal artery (not shown). This helical deployment enables continuous, circumferential treatment of nerve fibers surrounding the vessel. The helical geometry can be controlled through manipulation of internal guidewire positioning or refrigerant conduit rotation, allowing customization of the treatment pattern based on vessel anatomy and nerve fiber distribution. The deployed configuration maintains the atraumatic tip design while creating the expanded treatment geometry. The helical structure serves as an expansion chamber for refrigerant undergoing phase change and Joule-Thomson cooling while incorporating features to accommodate the deployable probe systems. In the configuration of FIGS. 1-4, there are at least two thermocouples, one inside the helical cooling element 16 and one on the atraumatic tip 18 (not shown). Exemplary probes are shown and described below.

FIG. 3 illustrates the adjustable capability of the helical configuration, showing how the pitch, outer diameter, and axial length can be modified during an ablation procedure. The arrows indicate the directional control capabilities that allow the operator to optimize the helical geometry for different vessel sizes and anatomical configurations. The helical structure can be expanded to vessel diameters typically ranging from 3-8 mm while maintaining wall contact for heat transfer and nerve ablation. The ability to adjust pitch and spacing allows for optimization of treatment density around the vessel circumference.

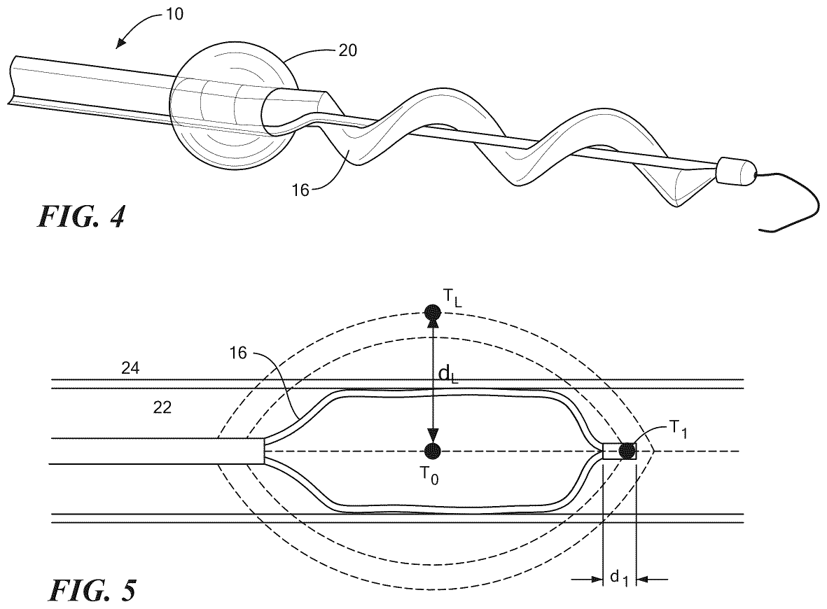

FIG. 4 shows an embodiment that incorporates a compliant occlusion balloon 20 positioned proximal to the heat transfer element 16. The occlusion balloon 20 provides vessel sealing capabilities when the heat transfer element alone does not achieve complete blood flow cessation. The occlusion balloon 20 can be inflated with saline or contrast solution and works with the treatment element to eliminate heat sink effects from residual blood flow. The dual occlusion approach provides treatment conditions while maintaining compatibility with the deployable probe systems. The positioning of the occlusion balloon proximally to the heat transfer element allows for independent control of vessel occlusion while maintaining the treatment capabilities of the primary helical element. The compliant occlusion balloon 20 is welded or bonded to the proximal shaft and it is deployed from its deflated position, on the outer surface of the proximal shaft by injecting saline or contrast-laced saline through a dedicated inflation lumen (not shown).

FIG. 5 provides a cross-sectional illustration of a temperature monitoring system and its relationship to the vessel anatomy and target nerve structures. The drawing shows an embodiment of a balloon-like treatment element 16 within a lumen 22 defined by a blood vessel 24, with temperature sensors T₀ positioned inside the treatment element and T₁ positioned at a known distance d₁ outside the treatment element 16. Additional temperature sensors can be positioned on the treatment element outer surface or proximal to it on the catheter shaft. The dashed lines represent isotherms of increasing temperature extending outward from the treatment element 16, with a lethal isotherm boundary calculated based on the temperature measurements and heat transfer modeling. This spatial arrangement enables calculation of the treatment depth and nerve ablation at target locations outside the vessel wall. The geometric relationship between the sensor positions and the calculated treatment zones provides the foundation for the computational algorithms that govern treatment progression and endpoint determination. The system maintains a database of tissue thermal properties specific to renal artery and perivascular tissue structures, enabling heat transfer calculations.

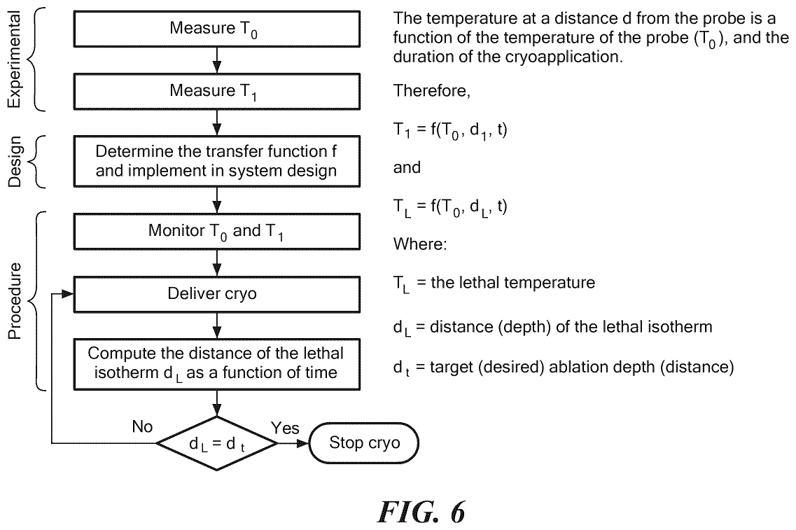

FIG. 6 is a flowchart that details the computational process governing the treatment monitoring and control. The process begins with experimental baseline measurements of temperatures T₀ and T₁, followed by determination of heat transfer functions. During a medical procedure using the catheter, the temperatures T₀ and T₁ are continuously monitored while delivering cryogenic energy (withdrawing heat from tissue). The measurements are calculated to compute the distance of the lethal isotherm d₁ as a function of time, using the relationship T₁=f(T₀, d₁, t) where temperature T₁ depends on probe temperature T₀, distance d₁, and time t. The system also incorporates the relationship T_{L}=f(T₀, d₁, t) where Tₜ represents the lethal temperature threshold. The calculated lethal isotherm distance d_{L} is compared with the target treatment depth dt, automatically determining when to stop cryogenic fluid delivery when the condition d_{L}=dt is satisfied. This closed-loop control system provides automated treatment endpoints compared to time-based or operator-judgment endpoints used in existing cryoablation approaches.

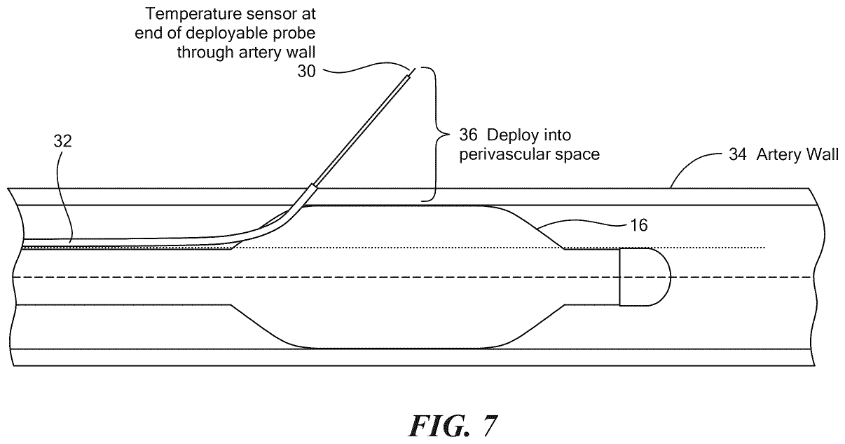

FIG. 7 illustrates elements that enable direct tissue temperature measurement at target nerve locations. A temperature sensor 30 is provided at the distal end of a deployable probe 32 extending through a blood vessel wall 34 into the perivascular space 36 where sympathetic nerves are located. The probe 32 allows for direct measurement of tissue temperature at a target (treatment) site, providing verification of nerve tissue temperature and enabling confirmation of ablation at the treatment location. This direct measurement capability addresses a limitation of existing cryoablation systems that infer treatment effectiveness through indirect measurements. A probe deployment system (not shown) includes mechanisms for controlled advancement and retraction while maintaining atraumatic tissue penetration, ensuring safe passage through vessel wall structures while maintaining temperature measurement accuracy. The probes are configured with sufficient length and flexibility to reach nerve tissue locations typically situated 1-10 mm from the vessel wall 34.

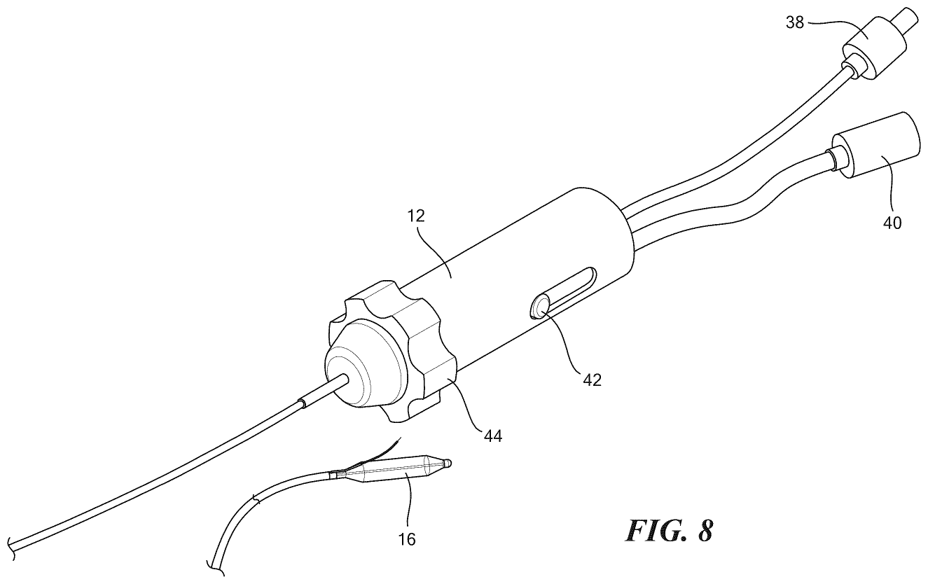

FIG. 8 shows a catheter handle 12 for use with the elements shown in FIG. 7, that integrates control mechanisms required for the monitoring and treatment delivery systems. The handle 12 includes connections for refrigerant supply and return 38, electrical connections 40 for the temperature monitoring systems, controls for the deployable probe mechanisms 42, and the deflection system 44 that enables catheter/shaft/treatment element positioning. The handle can be provided with display capabilities for temperature data and computational results.

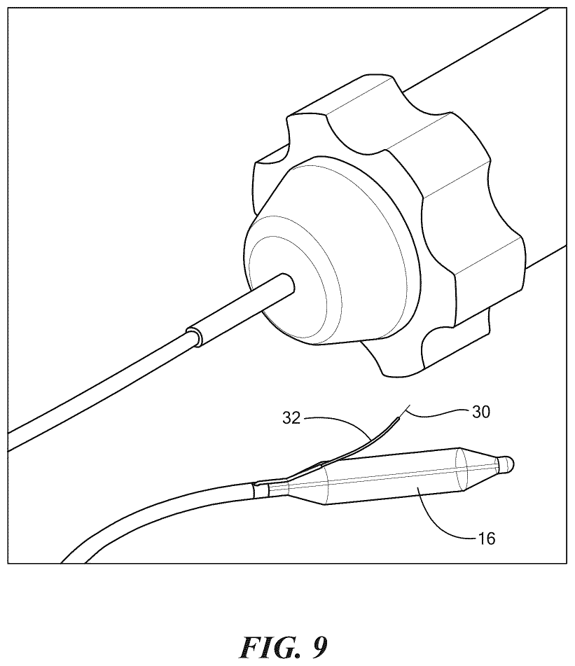

FIG. 9 is an enlarged view of the elements of FIGS. 7 and 8 with the integrated temperature probe 32 in the deployed position. This view shows the spatial relationship between the treatment element 16, the deployable probe 32 extending into the tissue, and the temperature sensor. The integration of the probe with the treatment element enables simultaneous treatment delivery and direct tissue temperature verification without compromising the effectiveness of either. The deployed probe 32 is shown extending beyond the treatment element to reach the target nerve tissue locations, providing direct temperature feedback from the ablation site. The probe positioning is guided by real-time imaging and controlled by the operator through dedicated controls integrated into the catheter hub. Multiple probes may be deployed simultaneously to provide temperature mapping of the treatment zone.

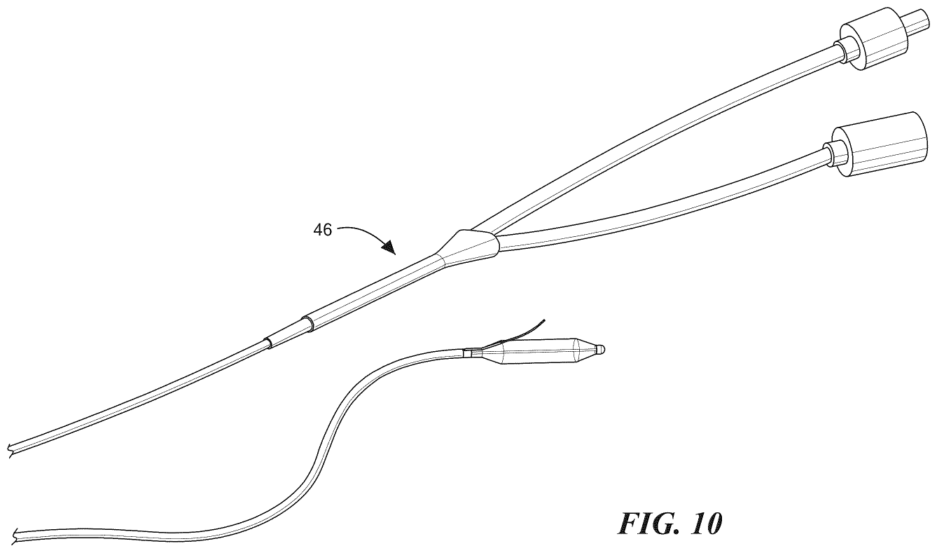

FIG. 10 shows a catheter 46 featuring a non-deflectable configuration with a manifold-type handle. This embodiment provides monitoring and treatment capabilities while offering a streamlined construction suitable for clinical applications where active deflection is not required. The manifold handle provides organized connections for the system components while maintaining monitoring capabilities. This configuration demonstrates the adaptability of the technology to different procedural requirements and operator preferences.

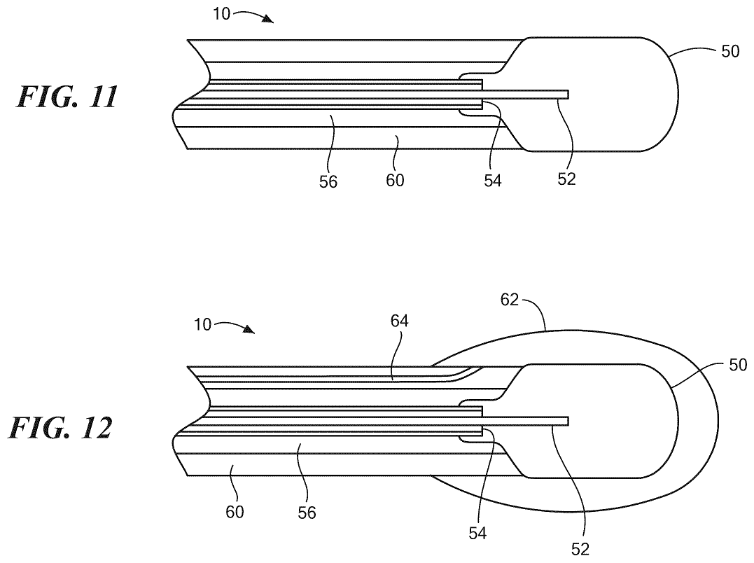

FIG. 11 illustrates a catheter 10 featuring a rigid tip for use with high-pressure refrigerant systems. In this embodiment, the rigid distal tip is a treatment element 50. A refrigerant tube 52 injects coolant into the tip 50, and a return tube 54 provides a flow path for coolant to the proximal end of the catheter. Insulation 56 is shown surrounding the return tube 54, and an external layer 60 surrounds the insulation. This configuration enables cooling performance using higher pressure refrigerant delivery, potentially providing deeper tissue penetration and more effective nerve ablation. Advanced embodiments utilize high-pressure refrigerant circulation, where liquid refrigerant is circulated under pressure to the cooling element and returned as liquid or gas/liquid mixture.

FIG. 12 depicts the catheter 10 of FIG. 11 with an optional inflatable balloon 62 covering the treatment element 50. The balloon 62 serves multiple functions including protection of blood vessel walls from direct contact with the high-pressure treatment element, creation of a controlled treatment environment, and potential for fluid filling to optimize heat transfer characteristics. The balloon material is selected for thermal conductivity and mechanical properties under cryogenic conditions while allowing for probe deployment through specialized ports.

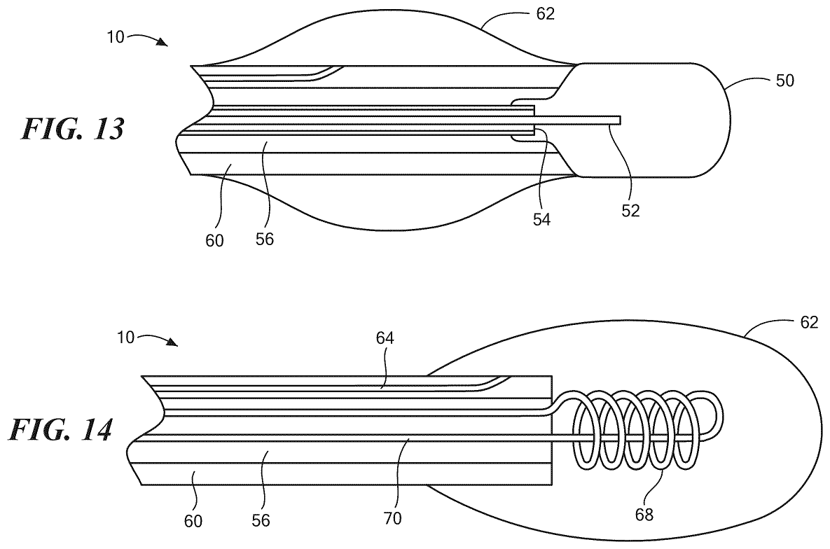

FIG. 13 shows the catheter 10 of FIG. 11 with a proximal occlusion balloon 62 for blood flow control. This eliminates heat sink effects in anatomical situations where single element occlusion, like the one in FIG. 4, may be insufficient. The proximal occlusion balloon 62 can be independently controlled to achieve vessel sealing regardless of the treatment element configuration.

FIG. 14 illustrates another configuration for the treatment element, wherein a balloon 62 surrounds a high-pressure helical treatment element 68. A lumen 64 is provided to inflate and deflate the balloon 62. In this configuration the coolant circulates through a helical tube rather than expanding within a balloon or rigid tip to facilitate extended treatment periods while maintaining compatibility with monitoring systems. A movable injection conduit 70 is provided, wherein longitudinal movement of the conduit alters the length, pitch and diameter of the helical treatment element 68.

The temperature monitoring system incorporating both fixed sensors and deployable probes provides continuous, real-time monitoring of tissue being treated. The deployable probe based system provides direct measurement of tissue temperature at target ablation sites outside the vessel wall, enabling verification of nerve ablation progress. The combined sensor data is processed by computational algorithms that calculate the distance of the lethal isotherm typically ranging from −20° C. to −40° C. from the treatment element surface based on measured temperatures, heat transfer models, and tissue property databases. Temperature data from all sensors is continuously processed and displayed to the operator in real-time, providing guidance for treatment duration and intensity adjustments. The system provides automated alerts when target temperatures are achieved at deployable probe locations, indicating nerve ablation.

The computational control system enables optimization of treatment parameters. The system incorporates algorithms that process temperature data from multiple sensor sources, apply heat transfer modeling based on tissue properties and geometric configurations, and provide calculation of lethal isotherm progression throughout the treatment zone. The algorithms account for factors including refrigerant temperature, flow rates, vessel occlusion status, blood flow effects, tissue thermal properties, and probe location data. The system provides predictive modeling of treatment progression, enabling operators to anticipate treatment endpoints and optimize procedure duration. The computational system interfaces with the catheter control mechanisms to provide integrated feedback on treatment element positioning, occlusion effectiveness, and probe deployment status.

The refrigerant/coolant delivery system is configured to provide control over cryogenic energy delivery while maintaining safety and reliability. A refrigerant supply/injection tube extends through the catheter shaft length, terminating in the treatment element region. The tube may feature single or multiple delivery ports positioned to optimize refrigerant distribution while accommodating probe deployment mechanisms. The treatment element can function as an expansion chamber where liquid refrigerant undergoes phase change and Joule-Thomson expansion, extracting heat from surrounding tissues. The system includes pressure regulation and safety features to prevent over-pressurization while maintaining cooling performance. The refrigerant system is integrated with the computational control system to enable automated adjustment of cooling parameters based on temperature feedback from deployable probes.

The system is compatible with multiple imaging modalities to provide procedural guidance and outcome assessment. Strategic placement of radiopaque markers enables fluoroscopic visualization of the treatment element position, temperature probe locations, and catheter tip. The system is compatible with intravascular ultrasound or external ultrasound imaging to visualize treatment progress and measure frozen tissue volume. Ultrasound imaging can provide visualization of ice ball formation and progression, offering complementary data to the temperature probe measurements. The imaging integration enables verification of probe placement relative to target nerve structures and confirmation of treatment zone geometry.

The catheter incorporates navigation features to facilitate positioning in complex vascular anatomy. A metallic core wire encapsulated in an atraumatic polymer body allows manual pre-shaping of the catheter tip to desired angles or curvatures. Active deflection systems enable catheter tip manipulation during the procedure, facilitating engagement with target vessel segments and branch vessels. The system accommodates standard guidewire techniques for navigation and positioning workflows while providing control capabilities. Alternative embodiments feature rapid exchange designs for device exchange and reduced procedure complexity.

The invention encompasses methods for performing cryo-ablation procedures with integrated monitoring and feedback. Standard vascular access techniques are used to introduce the catheter system, with navigation guided by fluoroscopy and optional intravascular imaging. The treatment element is positioned at the target location using anatomical landmarks, imaging guidance, and radiopaque markers. The treatment element is transitioned from its delivery configuration to the deployed shape based on vessel anatomy and treatment requirements. Vessel occlusion is achieved through treatment element expansion and optional occlusion balloon inflation, with confirmation via pressure monitoring and imaging. Deployable temperature probes are advanced through the vessel wall to target tissue locations for direct temperature measurement at predetermined sites corresponding to nerve fiber locations. Refrigerant delivery commences with monitoring of temperature from all sensor sources, pressure parameters, and optional imaging data. The computational control system continuously processes all data sources to calculate lethal isotherm progression and provide guidance to the operator. Treatment progression is monitored through direct temperature measurements at probe locations, providing confirmation of nerve ablation at target sites. Automated or manual termination occurs upon reaching target parameters as confirmed by deployable probe measurements and computational analysis. Following treatment completion, deployable probes are retracted, all components are returned to delivery configuration, and the system is withdrawn using standard techniques.

Multiple safety mechanisms are integrated throughout the system design. Automated temperature monitoring from deployable probes prevents excessive cooling that could cause tissue damage at nerve target sites. Safety valves and pressure monitoring prevent over-pressurization of the system. The deployable probe system includes safety mechanisms to prevent excessive probe advancement or tissue damage during deployment. Rapid system shutdown capabilities enable immediate treatment termination if complications arise. The computational control system provides continuous safety monitoring and automated alerts for parameter excursions. The reversible nature of early-stage cryogenic exposure provides a safety margin compared to irreversible thermal damage.

The system is suitable for renal denervation in resistant hypertension and may be applied to other conditions involving sympathetic nervous system hyperactivity. The deployable probe technology enables verification of nerve ablation, potentially improving treatment success rates compared to existing methods. Sympathetic denervation may provide benefit in heart failure patients with sympathetic activation, with the monitoring ensuring complete denervation. Modulation of renal sympathetic activity may improve metabolic parameters including glucose control and insulin sensitivity in metabolic syndrome, with probe-based confirmation of treatment success. The system may be applicable to other conditions where sympathetic nervous system modulation provides therapeutic benefit, with the monitoring capabilities enabling optimization of treatment parameters for various clinical applications.

What is claimed is:

1. A cryo-ablation system for treating resistant hypertension, comprising:
a cryo-ablation balloon configured to be placed within a blood vessel;
a deployable temperature probe outside the cryo-ablation catheter, to perform direct temperature measurement of tissue, at a known distance from the balloon.

2. The cryo-ablation catheter of claim 1, including an occlusive cryo-ablation balloon configured to be placed within a blood vessel;
the occlusive cryo-ablation balloon being selectively transitionable from a substantially flat configuration to a cylindrical, conical, or helical shape for creating a continuous, circumferential, or helical elongated cryogenic treatment region of at least 5 mm in length;
a temperature monitoring system configured to measure temperatures at the temperature probe; and
a pressure sensing system to detect arterial occlusion distal to the cryo-ablation balloon.

3. The cryo-ablation system of claim 1, wherein the catheter is an over-the-wire, rapid exchange, deflectable, or shapable configuration.

4. The cryo-ablation system of claim 1, further comprising a shapeable structure including a metallic core wire encapsulated in an atraumatic polymer body, allowing manual shaping of the catheter at a desired angle or curvature.

5. The cryo-ablation system of claim 1, further including a compliant occlusion balloon located on the catheter shaft proximal to the cryo-ablation balloon, wherein the occlusion balloon is configured to be manually inflated by the operator using a saline or contrast solution.

6. The cryo-ablation system of claim 1, wherein the cryo-ablation balloon is configured to transition to a helical shape.

7. The cryo-ablation system of claim 6, further comprising means to control the pitch, outer diameter, and length of the cryo-ablation balloon when deployed in the helical shape.

8. The cryo-ablation system of claim 1, further comprising an ultrasound imaging system to visualize progression of tissue freezing, measure a diameter of frozen tissue volume, and calculate a position of a lethal isotherm to provide real-time feedback during a cryo-ablation procedure.

9. The cryo-ablation system of claim 2, wherein the temperature monitoring system includes one or more temperature probes, at least one of which is situated outside the cryo-ablation balloon at a known distance to compute the distance of a lethal isotherm from the balloon surface.

10. The cryo-ablation system of claim 9, wherein the temperature monitoring system is configured to display the distance of the lethal isotherm as a guide during the cryo-ablation procedure.

11. The cryo-ablation system of claim 2, wherein the temperature monitoring system includes one or more deployable temperature probes that are advanceable through a vessel wall 1-10 mm.

12. The cryo-ablation system of claim 1, wherein the cryo-ablation balloon has a deployed diameter sufficient to completely occlude the blood vessel.

13. A method for treating resistant hypertension, comprising:
introducing a cryo-ablation catheter with an occlusive cryo-ablation balloon into a renal artery;
transitioning the cryo-ablation balloon from a flat configuration to a cylindrical, conical, or helical configuration within the renal artery to create a continuous cryogenic treatment region;
occluding the renal artery using the cryo-ablation balloon to reduce blood flow and increase the efficacy of the cryo-ablation;
achieving cryogenic temperatures to ablate renal sympathetic nerves adjacent to the renal artery;
measuring the temperature at a known distances outside the cryo-ablation balloon to determine tissue freezing; and
monitoring progression of tissue freezing at the known distances and adjusting cryo-ablation parameters based on real-time feedback from the temperature measurements.

14. The method of claim 13, further comprising using ultrasound to monitor progression of tissue freezing and using a pressure sensing system to confirm arterial occlusion distal to the cryo-ablation balloon.

15. The method of claim 14, further comprising manually shaping the cryo-ablation catheter to navigate and engage the distal branches of the renal artery.

16. The method of claim 14, further comprising the step of inflating a compliant occlusion balloon proximal to the cryo-ablation balloon to achieve vessel occlusion if the cryo-ablation balloon does not fully occlude the renal artery.

17. The method of claim 14, further comprising the step of computing a distance of a lethal isotherm from the cryo-ablation balloon and displaying this information to an operator for guiding duration and extent of cryo-ablation.

18. The method of claim 13, wherein cryo-ablation is guided by an ultrasound imaging externally or within the renal artery to visualize the progression of tissue freezing and provide real-time feedback for the procedure.

19. The method of claim 13, wherein the cryo-ablation is performed to treat medical conditions selected from the group consisting of resistant hypertension, heart failure, central sympathetic hyperactivity, metabolic syndrome, and obesity by cryo-ablating targets proximate to the renal artery.
